SEQUENCE LISTING (4 sequences)

SEQ ID NO: 1 - DNA, 480 bp, Artificial Sequence
  Mus musculus
atgagtattcagggtaccagtctgctgacccaaagtccggcaagtctgagcacctataacgatcagagcgttagctttgtcctggaaaacggttgctacgtcatcaacgttgacgatagcggtaaagaccaggaacaggatcaggttctgctgcgttattacgaaagtccgtgtccggcaagtcaatctggcgacggcgttgacggcaaaaaagtcatggtcaacatgagcccgatcaaagacaccgatatctggctgcacgcgaacgacaaagattattctgttgaactgcaacgcggcgacgttagtccgccggaacaggcgtttttcgtgctgcacaaaaaatccagcgacttcgtctccttcgagtgcaaaaatctgccgggtacctacatcggcgttaaagataaccagctggcactggtcgaagaaaaagacgagagctgcaacaacatcatgttcaaactgagcaaaatctaa

SEQ ID NO: 2 - DNA, 483 bp, Artificial Sequence
  Homo sapiens
atgagtattaccggcatcagcccgattaccgaatatctggcaagcctgagcacctacaacgatcaaagcatcacctttgcgctggaagacgaaagctacgagatctacgtcgaggacctgaaaaaagacgagaaaaaagacaaagtcctgctgagctactacgaaagccagcatccgagtaacgaatctggcgacggggttgacggtaaaatgctgatggttaccctgagtccgaccaaagatttctggctgcacgcgaacaacaaagaacacagcgtcgaactgcacaaatgcgaaaaaccgctgccggatcaggcgtttttcgtgctgcataacatgcacagcaactgcgtctcctttgagtgcaaaaccgatccgggcgtttttattggcgtcaaagacaaccacctggcgctgatcaaagttgatagctccgaaaacctgtgcaccgaaaacatcctgttcaaactgagcgagacctaa

SEQ ID NO: 3 - protein, 159 aa, Artificial Sequence
  Mus musculus
MSIQGTSLLTQSPASLSTYNDQSVSFVLENGCYVINVDDSGKDQEQDQVLLRYYESPCPASQSGDGVDGKKVMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 4 - protein, 160 aa, Artificial Sequence
  Homo sapiens
MSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

(19) United States
(12) Patent Application Publication
(10) Pub. No.: US 2023/0101029 A1
(43) Pub. Date: Mar. 30, 2023

(54) METHODS OF USING IL-33 PROTEIN IN TREATING CANCERS

(71) Applicant: GENERAL REGENERATIVES (SHANGHAI) LIMITED, Shanghai (CN)

(72) Inventors: Wei HAN, Shanghai (CN); Ping LUO, Shanghai (CN)

(21) Appl. No.: 17/767,952

(22) PCT Filed: Oct. 11, 2019
(86) PCT No.: PCT/CN2019/110605

§ 371 Date: Apr. 11, 2022

(51) Int. Cl. A61K 38/20; A61P 35/00; A61K 45/06

(52) U.S. Cl. CPC A61K 38/20; A61P 35/00; A61K 45/06

(57) ABSTRACT

Disclosed herein are use of IL-33 protein for the treatment, prevention, or reduction of onset or metastasis of a cancer by administering to a subject in need a therapeutically effective amount of IL-33 protein, such as human IL-33 protein, pharmaceutical compositions comprising IL-33 protein for treating cancer, and use of an agent capable of upregulating CD40/CD40L signaling pathway for treating cancer.

### TECHNICAL FIELD

The present disclosure relates to interleukin 33 (IL-33) proteins that have therapeutic uses. In particular, the present disclosure relates to methods of treating, preventing, or reducing onset or metastasis of a cancer by administering to a subject in need a therapeutically effective amount of IL-33 protein, such as human IL-33 protein.

### BACKGROUND

Cancers and tumors can be controlled or reduced by the immune system of a living body, such as human. The immune system includes several types of lymphoid and myeloid cells, e.g., monocytes, macrophages, dendritic cells (DCs), eosinophils, T cells, B cells, and neutrophils. These lymphoid and myeloid cells produce secreted signaling proteins known as cytokines. The cytokines include, e.g., interleukin-33 (IL-33), interferon-gamma (IFNγ), IL-12 and IL-23. Immune responses include, for example, inflammation, i.e., the accumulation of immune cells systemically or in a particular location of the body. In response to an infective agent or foreign substance, immune cells secrete cytokines, which, in turn, modulate immune cell proliferation, development, differentiation, or migration. Excessive immune response can produce pathological consequences, such as autoimmune disorders, whereas impaired immune response may result in cancer. Anti-tumor response by the immune system includes, for example, innate immunity, e.g., immunity that is mediated by macrophages, NK cells, and neutrophils; and adaptive immunity, e.g., immunity that is mediated by antigen presenting cells (APCs), T cells, and B cells (see, e.g., Abbas, et al. (eds.), Cellular and Molecular Immunology, W. B. Saunders Co., Philadelphia, Pa. (2000); Oppenheim and Feldmann (eds.), Cytokine Reference, Academic Press, San Diego, Calif. (2001); von Andrian and Mackay, New Engl. J. Med. 343:1020-1034 (2000); Davidson and Diamond, New Engl. J. Med. 345:340-350, (2001)).

Cytokines are powerful modulators of the immune response and have potential to dramatically affect the outcomes of immune-oncology therapeutic approaches. However, previous efforts to utilize cytokines in human subjects have yielded only modest efficacies and significant toxicities. Recent studies have suggested that a “targeted cytokine,” such as an antibody-cytokine fusion protein, may deliver cytokines to a desired cell type while minimizing peripheral exposure and thus toxicities (see, e.g., Guo et al., Cytokine Growth Factor Rev. 38:10-21 (2017); Jakobisiak M, et al., Cytokine Growth Factor Rev. 22(2):99-108 (2011); Robinson, T. & Schluns, K. S., Immunol. Lett. 190:159-168 (2017); Rhode et al., Cancer Immunol. Res. 4(1): 49-60 (2016); Conlon et al., J Clin. Oncol. 33(1): 74-82 (2015)). Accordingly, development of a therapeutic agent based on a targeted cytokine would be of great value in treatments of various diseases, such as cancers.

Interleukin IL-33, a member of the IL-1 family, is widely involved in the Th2-type immune response. IL-33 binds to its receptor complex consisting of ST2 (IL-1R-like-1) and IL-1 receptor accessory protein (IL-1RAcP). However, more and more evidence suggests that IL-33 functions to promote a Th1-type immune response, which is closely associated with tumor immunity (see, e.g., Schmitz et al., Immunity. 23:479-490 (2005); Baumann et al., Proc. Nat, Acad. Sci. 112:4056-4061 (2015); Komai-Koma et al., Immunobiology 221:412-417 (2016)).

Overexpression or injection of IL-33 reportedly significantly suppressed colon tumor growth (see, e.g. Eissmann et al., Can. Immu. Res. 6:409-421 (2018)). IL-33^{-/-} mice were more susceptible to colitis-associated cancer (see, e.g., Malik et al., J. Clin. Investigation. 126:4469-4481 (2016)), and knockdown of ST2 in CT26 colon tumor cells accelerated tumor growth (see, e.g., O'Donnell et al., Brit. J. Can. 114:37-43 (2016)). These findings indicate that IL-33 can delay colon tumor growth. Conversely, IL-33 was shown to exert a protumoral role in colon cancer (see, e.g. Li et al., J. Exp. Clin. Can, Res. CR. 37:196 (2018), an azoxymethane/dextran sodium sulfate model of colorectal cancer (CRC), and Ameri et al., Proc. Nat. Acad. Sci. 116:2646-2651 (2019)), and Apc^{min/+} mice (an animal model of human familial adenomatous polyposis) (see, e.g., Maywald et al., Proc. Nat. Acad. Sci. 112:E2487-2496 (2015)). This paradoxical effect has also been reported in breast cancer and a lung cancer model.

CD40 belongs to the tumor necrosis factor (TNF) receptor superfamily and is expressed on antigen-presenting cells, including dendritic cells (DCs), macrophages, monocytes, and B cells. The ligand for CD40 is CD40L, which is mainly expressed by activated (CD4⁺ and CD8⁺) T cells, activated NK cells, and activated platelets. Interaction between CD40L on CD4⁺ T cells and CD40 on DCs triggers the maturation of DCs, resulting in upregulation of major histocompatibility complex (MHC) and costimulatory expression, thereby facilitating the differentiation of naive CD4⁺ T and CD8⁺ T cells into helper T cells (Th) and cytotoxic T lymphocytes (CTLs), respectively. The related release of inflammatory cytokines indirectly leads to NK cell activation. Therefore, CD40/CD40L axis agonists are expected to improve the cancer immune response (see, e.g. Loskog et al., Endo. Meta. & Immu. Disorders-Drug Targets 7:23-28 (2007); Hassan et al., Immunophar. & Immunotox. 36:96-104 (2014); Vonderheide et al., Can. Cell 33:563-569 (2018)).

There is a need to develop a method of treating cancers using IL-33 protein.

### BRIEF SUMMARY

In one aspect, the present disclosure provides a method of treating, preventing, or reducing onset or metastasis of a cancer, comprising administering to a subject, such as human, in need a therapeutically effective amount of IL-33 protein, or a polypeptide having a corresponding sequence substantially identical thereto.

In one embodiment, the IL-33 protein is human IL-33.

In another embodiment, the human IL-33 is recombinant.

In certain embodiments, the human IL-33 has a sequence of SEQ ID NO:1.

In certain embodiments, the cancer disclosed herein is selected from the group consisting of a solid tumor selected from pancreatic cancer, small cell lung cancer (SCLC), hepatocellular carcinoma (HCC), squamous cell carcinoma, non-small cell lung cancer, squamous non-small cell lung cancer (NSCLC), non-squamous NSCLC, glioma, gastrointestinal cancer, renal cancer, ovarian cancer, liver cancer, colorectal cancer, endometrial cancer, kidney cancer, prostate cancer, thyroid cancer, neuroblastoma, glioblastoma, stomach cancer, bladder cancer, hepatoma, breast cancer, colon carcinoma, head and neck cancer, gastric cancer, germ cell tumor, pediatric sarcoma, sinonasal natural killer, melanoma, skin cancer, bone cancer, cervical cancer, uterine cancer, carcinoma of the fallopian tubes, carcinoma of the endometrium, carcinoma of the cervix, carcinoma of the vagina, carcinoma of the vulva, cancer of the anal region, testicular cancer, cancer of the esophagus, cancer of the small intestine, cancer of the endocrine system, cancer of the parathyroid gland, cancer of the adrenal gland, sarcoma of soft tissue, cancer of the urethra, cancer of the ureter, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus-related cancers, and cancers of viral origin; or a hematological cancer selected from acute lymphoblastic leukemia (ALL), acute myelogenous leukemia (AML), chronic lymphocytic leukemia (CLL), chronic myelogenous leukemia (CML), Hodgkin's lymphoma (HL), non-Hodgkin's lymphomas (NHLs), multiple myeloma, smoldering myeloma, monoclonal gammopathy of undetermined significance (MGUS), advanced, metastatic, refractory and/or recurrent hematological malignancies, and any combinations of said hematological malignancies.

In a further embodiment, the cancer is selected from the group consisting of hepatocellular carcinoma HOC lung cancer, gastric cancer, colon cancer, and prostate cancer.

In one embodiment, the cancer is hepatocellular carcinoma (HOC).

In another embodiment, the cancer is lung cancer.

In a further embodiment, the lung cancer is Lewis lung carcinoma.

In yet another embodiment, the cancer is gastric cancer.

In certain embodiments, the method further comprising administering with at least one anticancer entity.

In a further embodiment, the at least one anticancer entity is selected from the group consisting of a cytokine, an immunocytokine, TNFα, a PAP inhibitor, an oncolytic virus, a kinase inhibitor, an ALK inhibitor, a MEK inhibitor, an IDO inhibitor, a GLS1 inhibitor, a tyrosine kinase inhibitor, a CART cell or T cell therapy, a TLR agonist, a tumor vaccine, and an antibody selected, for example, from the group consisting of an anti-CTLA-4 antibody, an anti-CD3 antibody, an anti-CD4 antibody, an anti-CD8 antibody, an anti-4-1 BB antibody, an anti-PD-1 antibody, an anti-PD-L1 antibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSF1 R antibody, an anti-CSF1 antibody, an anti-IL-7R antibody, an anti-MARCO antibody, an anti-CXCR4 antibodies, an anti-VEGF antibody, an anti-VEGFR1 antibody, an anti-VEGFR2 antibody, an anti-TNFR1 antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD 52 antibody, an anti-CCR4 antibody, an anti-CCR8 antibody, an anti-CD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRG1 antibody, an anti-FLT3 antibody, an anti-B7-H4 antibody, an anti-B7-H3 antibody, an KLRG1 antibody, a BTN1A1 antibody, and an anti-GITR antibody.

In a second aspect, the present disclosure provides a composition comprising IL-33 protein or a polypeptide having a corresponding sequence substantially identical thereto as an active ingredient and at least one pharmaceutically acceptable carrier for use in treatment, prevention or reduction of onset or metastasis of a cancer.

In one embodiment, the IL-33 protein is human IL-33 protein.

In a third aspect, the present disclosure provides a method of treating, preventing, or reducing onset or metastasis of a cancer, comprising administering to a subject, such as human, in need a therapeutically effective amount of an agent capable of upregulating CD40/CD40L signaling pathway, or a polypeptide having a corresponding sequence substantially identical thereto.

In one embodiment, the agent capable of upregulating CD40/CD40L signaling pathway is IL-33 protein.

In a further embodiment, the IL-33 protein is human IL-33 protein.

In another embodiment, the human IL-33 is recombinant human IL-33.

In certain embodiments, the cancer is disclosed as set forth above.

### BRIEF DESCRIPTION OF THE DRAWINGS

FIG. 1 shows IL-33 protein inhibits Hepa 1-6 HOC growth.

FIGS. 2A and 2B show IL-33 protein suppresses LLC lung carcinoma growth,

FIG. 3 shows IL-33 protein inhibits MFC gastric cancer growth.

FIG. 4A and 4B show IL-33 protein restricts RM-1 prostate cancer growth.

FIGS. 5A and 5B show IL-33 treatment for murine colon cancer is time-dependent.

FIG. 6 shows the effect of IL-33 protein on murine colon cancer is affected by the initial treatment time.

FIGS. 7A to 7F show IL-33 protein significantly restrains CT26 mouse colon tumor growth and lung and liver metastasis.

FIGS. 8A to 8C show IL-33 protein activates multiple immune cells in vivo.

FIGS. 9A to 9C show CD4⁺ T cells, but not Tregs or eosinophils, are needed for IL-33 protein-induced antitumor immunity.

FIGS. 10A to 10D show that IL-33 protein promotes the expression of CD40L, CD40, and MHC-II on CD4⁺ T cells and DCs in the tumor microenvironment.

FIGS. 11A to 11C show that IL-33 protein has antitumor effects and activates CD4⁺ T, CD8⁺ T, and NK cells through CD40/CD40L signaling pathway.

FIGS. 12A to 12E show that IL-33 protein has antitumor activity via ST2 and stimulates CD4⁺ T cells to express ST2.

FIGS. 13A to 13E show endogenous IL-33 cannot boost antitumor immunity.

### DETAILED DESCRIPTION

The following terms, unless otherwise indicated, shall be understood to have the following meanings:

As used herein, including the claims, the singular forms of words, such as “a,” “an,” and “the,” include their corresponding plural references unless the context clearly dictates otherwise.

The terms “protein,”, “polypeptide,” and “peptide” are used interchangeably herein to refer to chains of amino acids of any length. The chain may be linear or branched, it may comprise modified amino acids, and/or may be interrupted by non-amino adds. The terms also encompass an amino add chain that has been modified naturally or by intervention; for example, disulfide bond formation, glycosylation, lipidation, acetylation, phosphorylation, or any other manipulation or modification, such as conjugation with a labeling component. Also such definition includes, for example, polypeptides containing one or more analogs of an amino acid (including, for example, unnatural amino adds, etc.), as well as other modifications known in the art. It is understood that the polypeptides can occur as single chains or associated chains.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)2, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody can be of any class, such as IgG, IgA, or IV (or sub-class thereof). Depending on the antibody's amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG-i, IgG2, IgG3, IgG4, IgAi and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

“Activity” of a molecule may refer, for example, to the binding of the molecule to a ligand or to a receptor, to catalytic activity; to the ability to stimulate gene expression or cell signaling, differentiation, or maturation; to antigenic activity, and to the modulation of activities of other molecules. “Activity” of a molecule may also refer to activity in modulating or maintaining cell-to-cell interactions, e.g., adhesion, or activity in maintaining a structure of a cell, e.g., cell membranes or cytoskeleton”.

“Administration” and “treatment”, as applied to an animal, human, experimental subject, cell, tissue, organ, or biological fluid, refers to contact of an exogenous pharmaceutical, therapeutic, diagnostic agent, compound, or composition to the animal, human, subject, cell, tissue, organ, or biological fluid. “Administration” and “treatment” can refer, e.g., to therapeutic, placebo, pharmacokinetic, diagnostic, research, and experimental methods. “Treatment of a cell” encompasses contact of a reagent to the cell, as well as contact of a reagent to a fluid, where the fluid is in contact with the cell. “Administration” and “treatment” also mean in vitro and ex vivo treatments, e.g., of a cell, by a reagent, diagnostic, binding composition, or by another cell, “Treatment,” as it applies to a human, veterinary, or research subject, refers to therapeutic treatment, prophylactic or preventative measures, to research and diagnostic applications.

The compositions and methods of the present disclosure encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

“Pharmaceutically effective amount” encompasses an amount sufficient to ameliorate or prevent a symptom or sign of the medical condition. A pharmaceutically effective amount also means an amount sufficient to allow or facilitate diagnosis. An effective amount for a particular patient or veterinary subject may vary depending on factors such as the condition being treated, the overall health of the patient, the method route and dose of administration and the severity of side effects. A pharmaceutically effective amount can be the maximal dose or dosing protocol that avoids significant side effects or toxic effects. The effect will result in an improvement of a diagnostic measure or parameter by at least 5%, such as by at least 10%, further such as at least 20%, further such as at least 30%, further such as at least 40%, further such as at least 50%, further such as at least 60%, further such as at least 70%, further such as at least 80%, and even further such as at least 90%, wherein 100% is defined as the diagnostic parameter shown by a normal subject. A pharmaceutically effective amount of IL-33 protein would be an amount that is, for example, sufficient to reduce a tumor volume, inhibit tumor growth, or prevent or reduce metastasis.

The term “pharmaceutically acceptable” refers to those compounds, materials, compositions, and/or dosage forms, which are suitable for use in contact with the tissues of human beings and animals without excessive toxicity, irritation, allergic response, or other problem or complication, commensurate with a reasonable benefit/risk ratio.

The term “subject” refers to a warm-blooded animal, such as a human that would benefit biologically, medically or in quality of life from the treatment. The subject can be mammals and non-mammals. Examples of the mammals include, but are not limited to, humans, chimpanzees, apes, monkeys, cattle, horses, sheep, goats, swine; rabbits, dogs, cats, rats, mice, guinea pigs, and the like. Examples of the non-mammals include, but are not limited to, birds, fish and the like. In one embodiment, the subject is human. It may be a human who has been diagnosed as in need of treatment for a disease or disorder disclosed herein.

“Exogenous” refers to substances that are produced outside an organism, cell, or human body, depending on the context. “Endogenous” refers to substances that are produced within a cell, organism, or human body, depending on the context.

“Anticancer entity” refers to any pharmaceutical entity that has an anticancer effect. The anticancer entity can be selected, for example, from a cytokine, an immunocytokine, TNFα, a PAP inhibitor, an oncolytic virus, a kinase inhibitor, an ALK inhibitor, a MEK inhibitor, an IDO inhibitor, a GLS1 inhibitor, a tyrosine kinase inhibitor, a CART cell or T cell therapy, a TLR agonist, or a tumor vaccine, or an antibody selected from the group consisting of an anti-CTLA-4 antibody, an anti-CD3 antibody, an anti-CD4 antibody, an anti-CD8 antibody, an anti-4-1 BB antibody, an anti-PD-1 antibody, an anti-PD-L1 antibody, an anti-T1M3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSF1 R antibody, an anti-CSF1 antibody, an anti-IL-7R antibody, an anti-MARCO antibody, an anti-CXCR4 antibodies, an anti-VEGF antibody, an anti-VEGFR1 antibody, an anti-VEGFR2 antibody, an anti-TNFR1 antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD 52 antibody, an anti-CCR4 antibody, an anti-CCR8 antibody, an anti-CD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRG1 antibody, an anti-FLT3 antibody, an anti-B7-H4 antibody, an anti-B7-H3 antibody, an KLRG1 antibody, a BTN1A1 antibody, and an anti-GITR antibody.

The present disclosure provides methods of treating proliferative disorders, e.g., a cancer, with an IL-33 protein. Specifically, IL-33 protein can improve the expression of CD40L and CD40 on CD4⁺ T cells and DCs, and therefore provides a significant improvement for cancer treatment.

Provided herein are IL-33 proteins that are capable of upregulating CD40/CD40L signaling pathway.

In one aspect, the present disclosure provides a mouse mature IL-33 (miL-33) nucleotide having the following sequence:

ATGAGTATTCAGGGTACCAGTCTGCTGACCCAAAG
TCCGGCAAGTCTGAGCACCTATAACGATCAGAGCG
TTAGCTTTGTCCTGGAAAACGGTTGCTACGTCATC
AACGTTGACGATAGCGGTAAAGACCAGGAACAGGA
TCAGGTTCTGCTGCGTTATTACGAAAGTCCGTGTC
CGGCAAGTCAATCTGGCGACGGCGTTGACGGCAAA
AAAGTCATGGTCAACATGAGCCCGATCAAAGACAC
CGATATCTGGCTGCACGCGAACGACAAAGATTATT
CTGTTGAACTGCAACGCGGCGACGTTAGTCCGCCG
GAACAGGCGTTTTTCGTGCTGCACAAAAAATCCAG
CGACTTCGTCTCCTTCGAGTGCAAAAATCTGCCGG
GTACCTACATCGGCGTTAAAGATAACCAGCTGGCA
CTGGTCGAAGAAAAAGACGAGAGCTGCAACAACAT
CATGTTCAAACTGAGCAAAATCTAA

As disclosed herein, in order to adapt mIL-33 to express in E. coli host, its coding sequence of is optimized and ATG (underlined) is added to its N-terminal.

In some embodiments, the mIL-33 comprises the following amino acid sequence:

MSIQGTSLLTQSPASLSTYNDQSVSFVLENGCYVI
NVDDSGKDQEQDQVLLRYYESPCPASQSGDGVDGK
KVMVNMSPIKDTDIWLHANDKDYSVELQRGDVSPP
EQAFFVLHKKSSDFVSFECKNLPGTYIGVKDNQLA
LVEEKDESCNNIMFKLSKI

In a second aspect, the present disclosure provides a human mature IL-33 (hIL-33) nucleotide having the following sequence:

ATGAGTATTACCGGCATCAGCCCGATTACCGAATA
TCTGGCAAGCCTGAGCACCTACAACGATCAAAGCA
TCACCTTTGCGCTGGAAGACGAAAGCTACGAGATC
TACGTCGAGGACCTGAAAAAAGACGAGAAAAAAGA
CAAAGTCCTGCTGAGCTACTACGAAAGCCAGCATC
CGAGTAACGAATCTGGCGACGGGGTTGACGGTAAA
ATGCTGATGGTTACCCTGAGTCCGACCAAAGATTT
CTGGCTGCACGCGAACAACAAAGAACACAGCGTCG
AACTGCACAAATGCGAAAAACCGCTGCCGGATCAG
GCGTTTTTCGTGCTGCATAACATGCACAGCAACTG
CGTCTCCTTTGAGTGCAAAACCGATCCGGGCGTTT
TTATTGGCGTCAAAGACAACCACCTGGCGCTGATC
AAAGTTGATAGCTCCGAAAACCTGTGCACCGAAAA
CATCCTGTTCAAACTGAGCGAGACCTAA

As disclosed herein, in order to adapt hIL-33 to express in E. coli host, its coding sequence of is optimized and ATG (underlined) is added to its N-terminal.

In some embodiments, the hIL-33 comprises the following amino acid sequence:

SEQ ID NO: 1
MSITGISPITEYLASLSTYNDQSITFALEDESYEI
YVEDLKKDEKKDKVLLSYYESQHPSNESGDGVDGK
MLMVTLSPTKDFWLHANNKEHSVELHKCEKPLPDQ
AFFVLHNMHSNCVSFECKTDPGVFIGVKDNHLALI
KVDSSENLCTENILFKLSET

The scope of the present disclosure is best understood with reference to the following examples, which are not intended to limit the present disclosure to the specific embodiments.

### Examples

1. IL-33 Treatment in Hepa 1-6 (Hepatocellular Carcinoma, HCC) Tumor-Bearing Mice

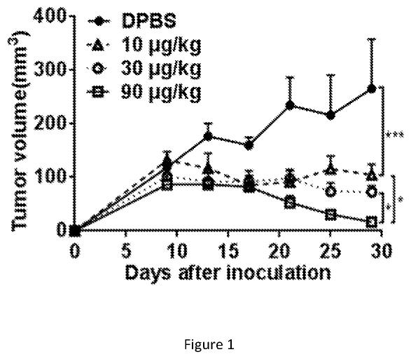

To evaluate the role of exogenous IL-33 protein on HOC, the dose-effect relationship research using Hepa 1-6 HOC model was carried out. In the Hepa 1-6 subcutaneous tumor-bearing mice model, the tumor volume of mice received 10, 30 or 90 μg/kg mIL-33 (recombinant IL-33) was much lower than that of DPBS (Dulbeco's phosphate buffered saline) solvent controls, respectively (P<0.001, FIG. 1). In addition, the tumor volume was significantly reduced in 90 μg/kg mIL-33 treatment group compared with 10 μg/kg or 30 μg/kg mIL-33 treatment group (P<0.05, FIG. 1). The results suggest that IL-33 protein efficiently suppresses murine Hepa 1-6 HOC growth, and such effect is dose-dependent, i.e., the antitumor effect is enhanced with the increase dose of mIL-33 protein.

FIG. 1 shows that IL-33 protein inhibits Hepa 1-6 HOC growth. C57BL/6 mice were injected subcutaneously with 4×10⁶ Hepa 1-6 HCC cells. 10 μg/kg, 30 μg/kg or 90 μg/kg mIL-33 protein was injected subcutaneously into mice respectively, once daily, starting from day 5 to the end of the test. Tumor volume was measured every 2 days, starting on day 7 after tumor cells inoculation. DPBS was the solvent control group and data are shown as the means±SEM (n=6 mice per group). *P<0.05, *** P<0.001.

2. IL-33 Treatment in LLC (Lewis Lung Carcinoma) Tumor-Bearing Mice

In the murine LLC lung carcinoma (belongs to non-small-cell lung cancer, NSCLC) metastasis model, the survival rate of IL-33 transgenic mice was significantly higher than that of the control group. However, the tumor growth showed a decreased trend by blocking of IL-33 in the human NSCLC tumor xenografts model.

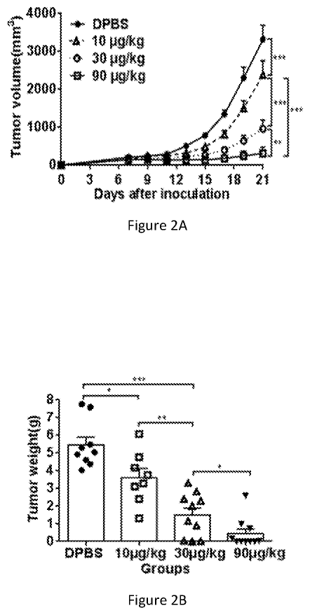

To further confirmed the effect of exogenous IL-33 protein on lung carcinoma, the dose-effect relationship research using LLC subcutaneous tumor-bearing mice model was performed. As shown in FIGS. 2A and 2B, the tumor volume and weight of mice injected with 30 or 90 μg/kg mIL-33 were much lower than that of DPBS group, respectively (volume, P<0.001; weight, P<0.001, FIGS. 2A and 2B). Simultaneously, the tumor volume and weight were markedly reduced in 10 μg/kg mIL-33 treatment group compared with DPBS group, respectively (volume, P<0.001; weight, P<0.05, FIGS. 2A and 2B). In addition, the tumor volume and weight were significantly reduced in 90 μg/kg mIL-33 treatment group (volume, P<0.01; weight, P<0.05, FIGS. 2A and 2B), but greatly increased in 10 μg/kg mIL-33 treatment group (volume, P<0.001; weight, P<0.01, FIGS. 2A and 2B) compared to 30 μg/kg mIL-33 treatment group. These data suggest that IL-33 protein significantly dampens murine LLC lung carcinoma growth, and such effect is dose-dependent, i.e., the antitumor activity is improved with the increase dose of mIL-33 protein.

FIGS. 2A and 2B suggest that IL-33 protein suppresses LLC lung carcinoma growth. C57BL/6 mice were injected subcutaneously with 4×10⁶ LLC lung carcinoma cells. 10 μg/kg, 30 μg/kg or 90 μg/kg mIL-33 protein was injected subcutaneously into mice respectively, once daily, starting from day 5 to the end of the test. Tumor volume was measured every 2 days, starting on day 7 after tumor cells inoculation. Mice were sacrificed at day 21 post the LLC inoculation and tumor tissues were acquired and weighed. DPBS was the solvent control group and data are shown as the means±SEM (n=8-10 mice per group). *P<0.05, **P<0.01, ***P<0.001.

3. IL-33 Treatment in MFC (Mouse Forestomach Carcinoma) Tumor Bearing Mice

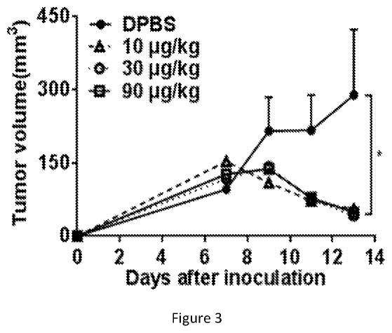

Correlative researches in the established MFC subcutaneous tumor-bearing mice model was performed. As shown in FIG. 3, the tumor volume was greatly reduced in those mice received 10, 30 or 90 μg/kg mIL-33 protein compared with DPBS solvent group (P<0.05, FIG. 3). The results suggest that IL-33 protein significantly restrains murine MFC gastric cancer growth and this inhibitory effect can be achieved at a related low level of IL-33 protein (10 μg/kg).

FIG. 3 shows that IL-33 protein inhibits MFC gastric cancer growth. BALB/c mice were injected subcutaneously with 4×10⁶ MFC gastric cancer cells. 10 μg/kg, 30 μg/kg or 90 μg/kg mIL-33 was injected subcutaneously into mice respectively, once daily, starting from day 5 to the end of the test. Tumor volume was measured every 2 days, starting on day 7 after tumor cells inoculation. DPBS was the solvent control group and data are shown as the means±SEM (n=9 mice per group). *P<0.05.

4. IL-33 Treatment in RM-1 (Prostate Cancer) Tumor-Bearing Mice

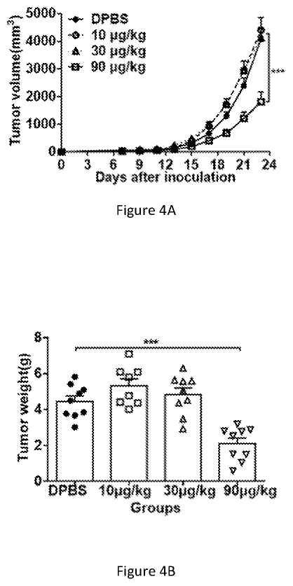

To evaluate the influence of IL-33 on prostate cancer, the dose-effect research utilizing RM-1 subcutaneous tumor-bearing mice model was carried out. It was found that the tumor volume and weight among DPBS solvent control group, 10 μg/kg mIL-33 and 30 μg/kg mIL-33 treatment group showed no significant difference, but both were markedly higher than that of 90 μg/kg mIL-33 treatment group (volume, P<0.001; weight, P<0.001, FIGS. 4A and 4B). The results suggest that IL-33 protein can significantly restrict RM-1 prostate cancer growth, but such antitumor effect needs to be exerted at a relatively high dose of IL-33 protein (90 μg/kg).

FIGS. 4A and 4B show that IL-33 protein restricts RM-1 prostate cancer growth. C57BL/6 mice were injected subcutaneously with 2×10⁶ RM-1 prostate cancer cells. 10 μg/kg, 30 μg/kg or 90 μg/kg mIL-33 protein was injected subcutaneously into mice respectively, once daily, starting from day 5 to the end of the test. Tumor volume was measured every 2 days, starting on day 7 after tumor cells inoculation. Mice were sacrificed at day 23 post RM-1 inoculation and tumor tissues were acquired and weighed. DPBS was the solvent control group and data are shown as the means±SEM (n=8-9 mice per group). *** P<0.001.

5. IL-33 Treatment for Murine Colon Cancer is Time-Dependent

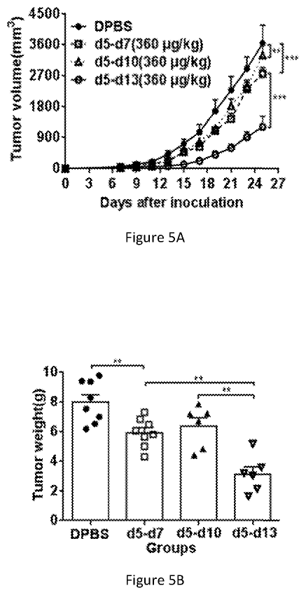

In the CT26 colon cancer subcutaneous tumor-bearing mice model, mIL-33 protein was injected on the day 5 after tumor cells inoculation. The days of administration were set as 3 days (d 5-d 7), 6 days (d 5 d 7) or 9 days (d 5-d 13), respectively. As shown in FIGS. 5A and 5B, the tumor volume and weight both were significantly enhanced in DPBS solvent group (volume, P<0.01; weight, P<0.01, FIGS. 5A and 5B), but markedly reduced in mIL-33 treatment group administered for 9 days (volume, P<0.001; weight, P<0.01, FIGS. 5A and 5B), compared with mIL-33 treatment group administered for 3 days or 6 days. These results show that IL-33 protein can efficiently inhibit murine colon cancer growth. Although the antitumor effect between mIL-33 treatment group administered for 3 days and mIL-33 treatment group administered for 6 days was of no significant difference, such inhibitory action was greatly increased when the treatment period was expanded to 9 days. The data indicate that IL-33 protein can quickly activate the antitumor immune response, but its intensity of action is time-dependent, i.e., such effect can be improved with the increased time of treatment.

FIGS. 5A and 5B show IL-33 treatment for murine colon cancer is time-dependent. BALB/c mice were injected subcutaneously with 1×10⁶ CT26 colon cancer cells. 360 μg/kg mIL-33 protein was injected subcutaneously into mice, once daily, starting from day 5 to day 7, day 10 or day 13, respectively. Tumor volume was measured every 2 days, starting on day 7 after tumor cells inoculation. Mice were sacrificed at day 27 post CT26 inoculation and tumor tissues were acquired and weighed. DPBS, injected during day 5 to day 13, was the solvent control group. Data are shown as the means±SEM (n=6-8 mice per group). **P<0.01, ***P<0.001.

6. The Effect of IL-33 on Murine Colon Cancer is Affected by the Initial Treatment Time

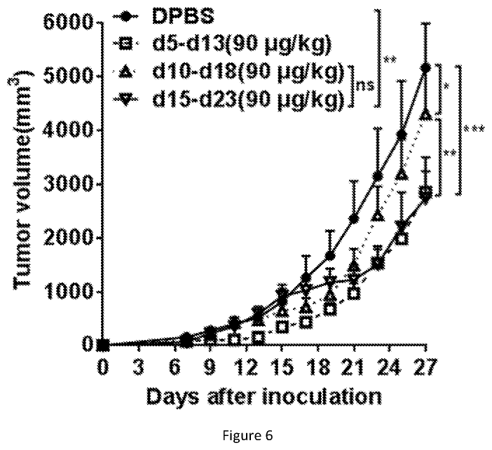

In the CT26 colon cancer subcutaneous tumor-bearing mice model, mIL-33 protein (90 μg/kg, once daily) was administered for 9 days, starting on the day 5 (d 5-d 13), day 10 (d 10-d 18) or day 15 (d 15-d 23), respectively. The tumor volume was significantly increased in DPBS solvent group (P<0.05, FIG. 6), but greatly reduced in mIL-33 treatment group administered from day 5 (P<0.01, FIG. 6), compared with mIL-33 treatment group administered from day 10. The tumor growth of mIL-33 treatment group injected from day 15 slowed down rapidly with mlL-33 administration and showed a similar trend after day 21 compared with mIL-33 treatment group injected from day 5, indicating that these two kinds of administration plans had the similar effect on the tumor growth. In addition, the tumor volume between mIL-33 treatment group administered from day 15 and mlL-33 treatment group administered from day 10 showed no significant difference, but there was a decreased trend in mIL-33 treatment group administered from day 15 (FIG. 6). These results indicated that the inhibitory effect of IL-33 protein on murine colon cancer was associated with the initial administration time. IL-33 protein activated the antitumor immune response more efficiently and durably at the “early stage” (day 5) or “later stage” (day 15) of tumor progression, but its antitumor effect was relatively weak in the “middle period” (day 10) of the tumor development.

FIG. 6 shows the effect of IL-33 protein on murine colon cancer is affected by the initial treatment time. BALB/c mice were injected subcutaneously with 1×10⁶ CT26 colon cancer cells. 90 μg/kg mIL-33 was injected subcutaneously into mice for 9 days, once daily, starting from day 5, day 10, or day 15, respectively. Tumor volume was measured every 2 days, starting on day 7 after tumor cells inoculation. DPBS, injected during day 5 to day 23, was the solvent control group. Data are shown as the means±SEM (n=7-8 mice per group). * P<0.05, ** P<0.01, *** P<0.001.

7. IL-33 Treatment Effectively Inhibits CT26 Mouse Colon Tumor Growth and Lung and Liver Metastasis

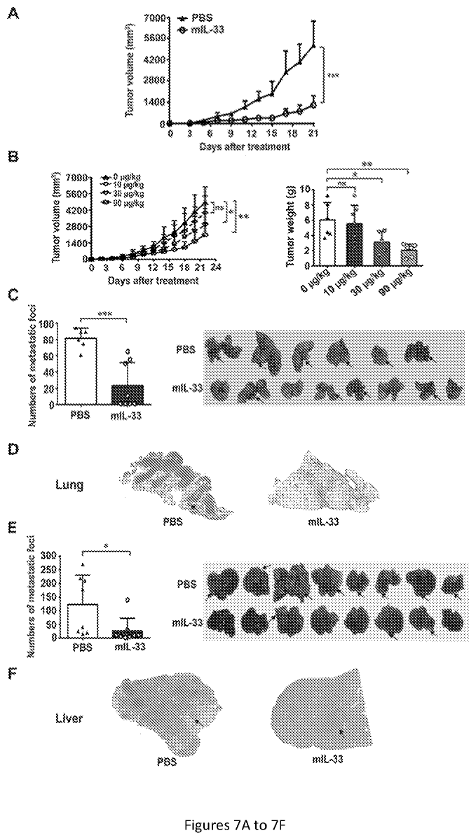

To clarify the effects of IL-33 on CT26 murine colon subcutaneous tumor, and pulmonary and liver metastasis, mIL-33 protein was subcutaneously injected into the mice on the day of CT26 cell injection in the subcutaneous tumor-bearing mouse model (FIG. 7A) and pulmonary metastasis model (FIGS. 7C, 7D). To prevent surgical wound infection caused by scratching of the wound by the mice, the time for the administration of mIL-33 protein in the liver metastasis model was delayed, starting on day 8 after CT26 inoculation (FIGS. 7E, 7F). In the dose-effect relationship experiments, mice received mIL-33 protein injection when tumor was visible (starting on day 5 after CT26 inoculation), which may be more meaningful for clinical applications (FIG. 7B).

In the CT26 subcutaneous tumor-bearing mouse model, the tumor growth rate in the mIL-33 protein group was significantly lower than that in the PBS (phosphate buffered saline) control group (P<0.001, FIG. 7A). The antitumor effect of IL-33 protein on CT26 subcutaneous colon cancer was confirmed using dose-effect relationship studies. It was found that IL-33 protein-mediated antitumor activity was dose-dependent. Tumor growth slowed down and tumor mass declined with an increasing dose of mIL-33 protein (FIG. 7B). In the CT26 pulmonary and liver metastasis models, the numbers of metastatic nodules at the surfaces of the lungs and liver were greatly reduced in the mIL-33 protein group compared with the PBS controls (pulmonary metastasis, P<0.001; liver metastasis, P<0.05; FIGS. 7C, 7E). This was confirmed by H&E staining analysis (FIGS. 7D, 7F). These data suggested that IL-33 protein could suppress the growth and pulmonary and liver metastasis of CT26 colon tumor cells.

FIGS. 7A to 7F show IL-33 protein significantly restrains CT26 mouse colon tumor growth and lung and liver metastasis. FIGS. 7A and 7B relate to subcutaneous CT26 tumor-bearing mouse model. FIGS. 7C and 7D relate to pulmonary metastasis model, wherein 7C shows numbers of visible tumor nodules (left panel) and photographs of metastatic lung tissues (right panel). FIG. 7D relates to representative photomicrographs of H&E-stained lung tissues (500 μm). FIGS. 7E and 7F relate to Liver metastasis model, wherein FIG. 7E shows numbers of visible tumor nodules (left panel) and photographs of metastatic liver tissues (right panel). FIG. 7F shows representative photomicrographs of H&E-stained liver tissues (500 μm). PBS-treated mice were used as the control group. Data are shown as means±SDs (n=5-8 mice per group). ns: no significant difference. *P<0.05, **P<0.01, ***P<0.001.

8. IL-33 Regulates Multiple Immune Responses

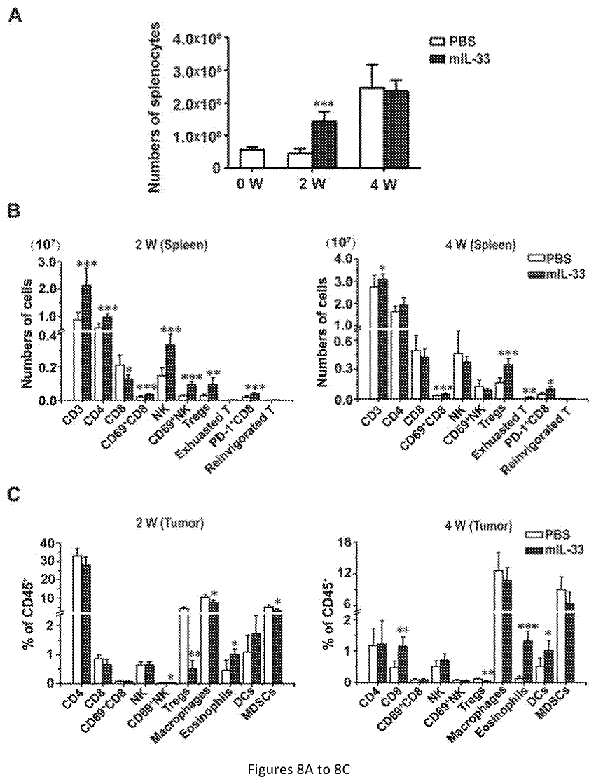

The effect of IL-33 on various immune cells, at various stages of tumor progression, and in spleen as well as tumor tissues, were evaluated. At 2 weeks after CT26 cell inoculation, significant splenomegaly was observed in the mIL-33 group (P<0.001, FIG. 8A). The numbers of splenic CD3⁺ T, CD4⁺ T, CD69⁺ CD8⁺ T (activated CD8⁺ T), NK, and CD69⁺ NK (activated NK) cells were greatly increased (P<0.001), whereas the numbers of splenic CD8⁺ T cells were significantly reduced in the mIL-33 group compared with the PBS group (P<0.05) (FIG. 8B, left panel). Additionally, mIL-33 protein significantly enhanced the numbers of splenic Tregs (P<0.01) and PD-1⁺ CD8⁺ T cells (P<0.001) (FIG. 8B, left panel). These data indicated that IL-33 had a proliferation and activation effect on multiple immune cells, such as on immune-system activation-related cells, when CT26 subcutaneous colon tumor developed at 2 weeks.

In the spleens of CT26 tumor-bearing mice at 4 weeks after inoculation, the numbers of CD3⁺ T (P<0.05) and CD69⁺ CD8⁺ T (P<0.001) cells were significantly higher in the mIL-33 group than in the PBS group (FIG. 8B, right panel). Similarly, mlL-33 protein treatment significantly increased the numbers of Tregs (P<0.001), exhausted CD8⁺ T cells (PD-1^{high}Eomes^{high}CD8⁺) (P<0.01), and PD-1⁺ CD8⁺ T cells (P<0.05) (FIG. 8B, right panel). These results showed that IL-33 activated CD8⁺ T cells when CT26 tumors developed at 4 weeks, but this positive simulation of the immune systems gradually weakened.

Then changes in various immune cells in the tumor microenvironment were investigated. At 2 weeks after CT26 cell inoculation, mIL-33-injected mice showed marked increases in the fractions of tumor-infiltrating CD69⁺ NK cells (P<0.05) and eosinophils (P<0.05) among CD45⁺ cells, but significant decreases in the fractions of tumor-infiltrating Tregs (P<0.01), macrophages (P<0.05), and myeloid-derived suppressor cells (MDSCs) (P<0.05) among CD45⁺ cells compared to PBS-injected mice (FIG. 8C, left panel). At 4 weeks after CT26 cells inoculation, mIL-33 protein significantly enhanced the fractions of tumor-infiltrating CD8⁺ T cells (P<0.01), eosinophils (P<0.001) and DCs (P<0.05) among CD45⁺ cells, whereas it greatly reduced the fraction of tumor-infiltrating Tregs (P<0.01) among CD45⁺ cells (FIG. 8C, right panel). These data suggested that, similar to the effect in spleens, IL-33 protein affected the composition ratio of multiple immune cells in the CT26 tumor microenvironment. When CT26 tumors developed at 2 weeks, the antitumor effect of IL-33 seemed to correspond to a decrease in Tregs and an increase in CD69⁺ NK cells. However, when tumor developed at 4 weeks, the fractions of CD8⁺ T cells and eosinophils changed more significantly than those of other immune cells.

IL-33 protein affected the numbers and fractions of multiple immune cells in spleen and tumor tissues.

FIGS. 8A to 8C show IL-33 protein activates multiple immune cells in vivo in subcutaneous CT26 tumor-bearing mouse model. Mice were sacrificed on day 0 (0 w), 14 (2 w), or 28 (4 w) post CT26 inoculation. FIG. 8A shows splenocyte numbers. FIG. 8B shows the flow-cytometric analysis of splenic immune cells. FIG. 8C shows the flow-cytometric analysis of tumor-infiltrating immune cells. PBS-injected mice served as the control group. Exhausted T cells are PD-1^{high}Eomes^{high}CD8⁺ and reinvigorated T cells are PD-1^{mid}T-bet^{high}CD8⁺. Data are shown as means±SDs (n=5-6 mice per group). *P<0.05, **P<0.01, ***P<0.001.

9. CD4⁺ T Cells, but not Tregs or Eosinophils, Play an Important Role in IL-33-Mediated Antitumor Effects

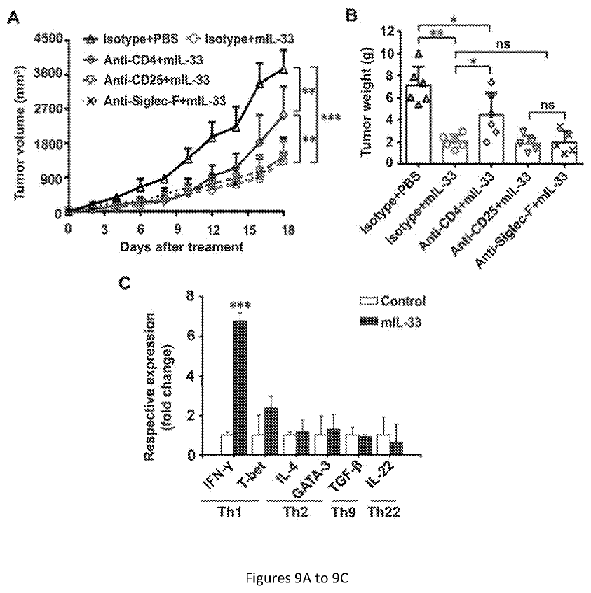

To clarify the relationship between the antitumor activity of IL-33 protein and CD4⁺ T cells, Tregs, or eosinophils, these cells in vivo using antibodies were depleted. The results showed that tumor growth and mass of isotype of anti-CD4 antibody (isotype)+mIL-33 group were lower than those in the anti-CD4 antibody (anti-CD4)+mIL-33 group (volume, P<0.01; weight, P<0.05), but there was no significant difference with tumor growth and mass in the anti-CD25 antibody (specific depletion of Tregs (anti-CD25)+mIL-33 group or anti-Siglec-F antibody (specific depletion of eosinophils) (anti-Siglec-F)+mIL-33 group (FIG. 9A, 9B). These data indicated that CD4⁺ T cells played an important role in IL-33 protein-mediated antitumor immunity, whereas Tregs and eosinophils contribute little to these effects. Notably, tumor growth (P<0.01) and mass (P<0.05) in the anti-CD4+mIL-33 group were lower than those in the isotype+PBS group (FIG. 9A, 9B). Therefore, IL-33 protein exerted its antitumor effect through other signals, in addition to CD4⁺ T cells.

CD4⁺ T cells are divided into several subtypes based on the transcription factors and cytokines they express. Different subtypes of CD4⁺ T cells have different functions in cancer immunity. Thus, which types of CD4⁺ T cells IL-33 protein acts on, using RT-qPCR, was investigated. As shown in FIG. 9C, the expression level of IFN-γ in the mIL-33 group was drastically higher than that in the control group (P<0.001), and T-bet also tended to be upregulated. However, the expression levels of IL-4, GATA-3, TGF-β, and IL-22 showed no difference between the above two groups. These results suggested that IL-33 protein may promote the activation of Th1, but not Th2, Th9, and Th22 cells.

FIGS. 9A to 9C show CD4⁺ T cells, but not Tregs or eosinophils, are needed for IL-33 protein-induced antitumor immunity. FIGS. 9A-9C show subcutaneous CT26 tumor-bearing mouse model. Mice were sacrificed on day 19 post PBS or mlL-33 treatment. FIG. 9A shows tumor volumes. FIG. 9B shows tumor weights. FIG. 9C shows RT-qPCR analysis of mRNA expression of Th1-, Th2-, Th9-, and Th22-related cytokines and transcription factors in tumor tissues. Target gene expression was normalized to that of GAPDH. Isotype control of anti-CD4 antibody was included. Data are means±SDs (n=4-6 mice per group). ns: no significant difference. *P<0.05, **P<0.01, ***P<0.001.

10. IL-33 Regulates the Expression Levels of CD40L, CD40, and MHC-II on CD4+ T Cells and DCs in the Tumor Microenvironment

The effects of IL-33 protein on CD4⁺ T cells and DCs were further investigated.

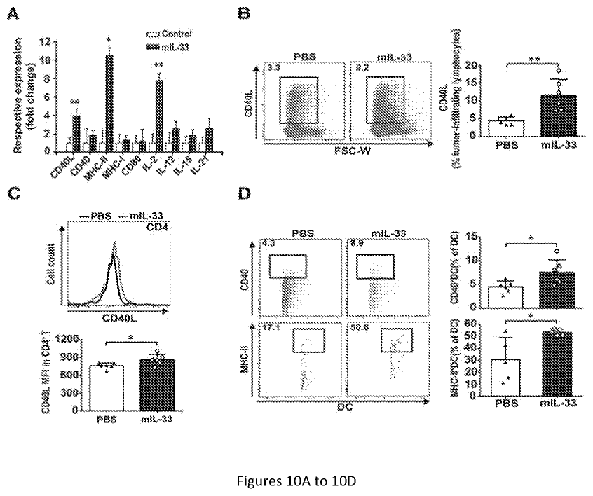

It was analyzed which types of MHC, costimulatory molecules and cytokines are affected by IL-33 protein using RT-qPCR and flow cytometry. The expression levels of CD40L (P<0.01), MHC-II (P<0.05), and IL-2 (P<0.01) from tumor tissues in the mIL-33 group were significantly higher than those in the control group (FIG. 10A). Compared to the PBS control group, the percentages of CD40L on tumor-infiltrating lymphocytes (P<0.01, FIG. 10B) and the MFI of CD40L on tumor-infiltrating CD4⁺ T cells were increased in the mIL-33 group (P<0.05, FIG. 10C). Similarly, the fractions of CD40 (P<0.05, FIG. 10D, upper panel) and MHC-II (P<0.05, FIG. 10D, lower panel) on tumor-infiltrating DCs in the mIL-33 group were significantly higher than those in the control PBS group. These results suggested that IL-33 protein participated in the immune activation of CD4⁺ T cells and DCs by regulating the expression levels of CD40L, CD40, and MHC-II.

FIGS. 10A to 10D show that IL-33 protein promotes the expression of CD40L, CD40, and MHC-II on CD4⁺ T cells and DCs in the tumor microenvironment. FIG. 10A-10D show subcutaneous CT26 tumor-bearing mouse model. Mice were sacrificed on day 19 post PBS or mIL-33 treatment. FIG. 10A shows RT-qPCR analysis of CD40L, CD40, MHC-II, MHC-I, CD80, IL-2, IL-12, IL-15, and IL-21 mRNA expression in tumor tissues. Target gene expression was normalized to that of GAPDH. FIG. 10B shows flow-cytometric analysis of CD40L expression on tumor-infiltrating lymphocytes. Representative dot plots (left panel) and quantitative data (right panel). FIG. 100 shows MFI of CD40L on tumor-infiltrating CD4⁺ T cells. Representative histograms (upper panel), quantitative data (lower panel). FIG. 10D shows flow-cytometric analysis of CD40, and MHC-II expression on tumor-infiltrating DCs. Representative dot plots (left panel) and quantitative data (right panel). PBS was injected in control mice. Data are means±SD (n=4-6 mice per group). * P<0.05, **P<0.01.

11. Blockage of CD40/CD40L Signaling Attenuates the Antitumor Activity of IL-33

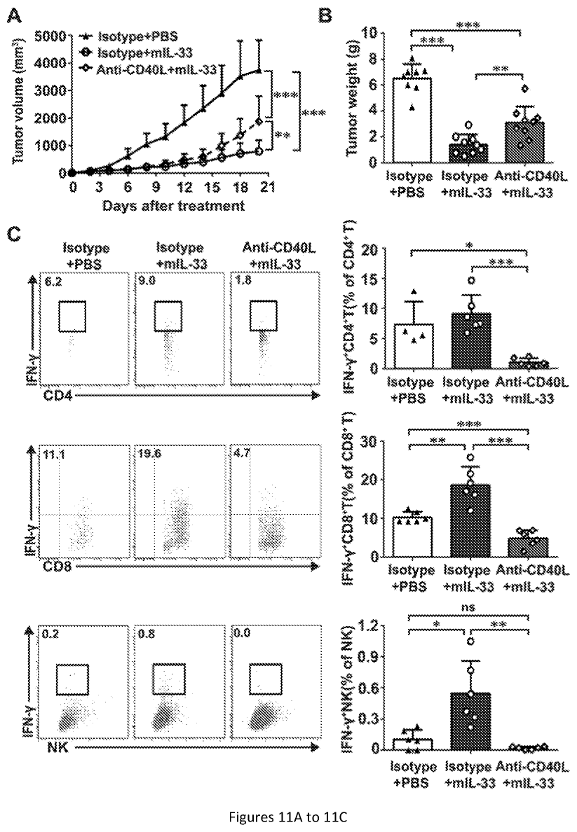

The effect of anti-CD40L antibody on IL-33 protein-induced antitumor activity was evaluated via neutralization experiments in vivo in mice. In CT26 tumor-bearing mice (FIG. 11A-11C), tumor growth (P<0.01, FIG. 11A) and mass (P<0.01, FIG. 11B) were significantly increased and the percentages of tumor-infiltrating IFN-γ⁺CD4⁺ T (P<0.001), IFN-γ⁺CD8⁺ T (P<0.001), and IFN-γ⁺NK (P<0.01) cells were markedly decreased (FIG. 11C) in the anti-CD40L+mIL-33 group compared with the isotype+mIL-33 group. These data indicated that the CD40/CD40L pathway was involved in IL-33 protein-mediated antitumor immunity and played a role in the activation of CD4⁺ T, CD8⁺ T, and NK cells induced by IL-33 protein.

Of note, tumor growth (P<0.001) and mass (P<0.001) in the anti-CD40L+mIL-33 group were significantly lower than those in the isotype+PBS group (FIG. 11A, 11B). However, the fractions of tumor-infiltrating IFN-γ+CD4⁺ T cells (P<0.05) and IFN-γ+CD8⁺ T cells (P<0.001) were significantly elevated in the isotype+PBS group compared to the anti-CD40L+mIL-33 group (FIG. 11C). Thus, IL-33 protein probably exerted its antitumor function via additional types of immune cells or signaling pathways.

FIGS. 11A to 11C show that IL-33 protein exerts antitumor effects and activates CD4⁺ T, CD8⁺ T, and NK cells through CD40/CD40L signaling pathway. FIGS. 11A-11C show subcutaneous CT26 tumor-bearing mouse model. Mice were sacrificed on day 21 post PBS or mIL-33 treatment. FIG. 11A shows tumor volumes. FIG. 11B shows tumor weights. FIG. 11C shows the flow-cytometric analysis of INF-γ expression on tumor-infiltrating CD4⁺ T, CD8⁺ T, and NK cells. Representative dot plots (left panel) and quantitative data (right panel). Isotype control of anti-CD40L antibody was included. Data are means±SD (n=4-9 mice per group). ns: no significant difference. * P<0.05, ** P<0.01, *** P<0.001.

### 12. Antitumor Immunity Effect of IL-33 is ST2-Dependent

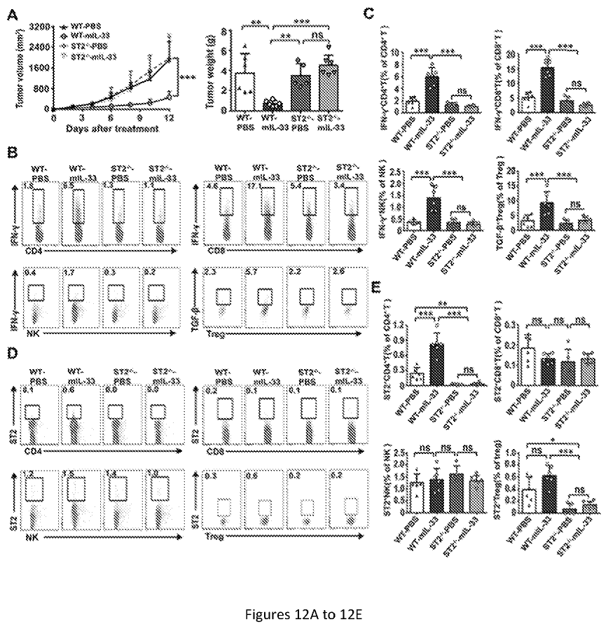

To determine whether the IL-33 protein-mediated antitumor activity and immune response are dependent on its natural receptor ST2, ST2^{-/-} tumor-bearing model mice was used to observe the antitumor effect of IL-33 protein. In the CT26 cell subcutaneous tumor-bearing model, it was found that tumor growth and mass were not significantly different among PBS-injected WT (WT-PBS), ST2^{-/-}-mIL-33, and ST2^{-/-}-PBS groups, but were significantly higher than in the WT-mIL-33 group (tumor growth, P<0.001; mass, P<0.01 or P<0.001; FIG. 12A). These results suggested that the antitumor effect of IL-33 protein was dependent on its receptor ST2.

The fractions of splenic IFN-γ⁺CD4⁺ T cells (P<0.001), IFN-γ⁺CD8⁺ T cells (P<0.001), IFN-γ⁺NK cells (P<0.001) and TGF-β⁺ Tregs (P<0.001) in the WT-mIL-33 group were higher than those in the WT-PBS group, whereas fractions of these cells showed no significant difference between the ST2^{-/-}-mIL-33 and ST2^{-/-}-PBS groups (FIGS. 12B and 12C). These data suggested that IL-33 protein activated CD4⁺ T cells, CD8⁺ T cells, NK cells, and Tregs depending on receptor ST2.

Further, the expression ratio of ST2 on splenic CD4⁺ T cells in the WT-PBS group was significantly higher than that in the ST2^{-/-}-PBS (P<0.01) and ST2^{-/-}-mIL-33 (P<0.01) groups, but significantly lower than that in the WT-mIL-33 group (P<0.001) (FIGS. 12D and 12E). These data indicated that ST2 was expressed by CD4⁺ T cells and was positively regulated by IL-33. Thus, it was speculated that IL-33 protein directly activated CD4⁺ T cells via ST2, and this output was gradually enhanced via a positive feedback loop. In addition, it was speculated that CD8⁺ T and NK cells hardly expressed ST2, and could be induced by IL-33 protein (WT-PBS vs. WT-mIL-33; ST2^{-/-}-PBS vs. ST2^{-/-}-mIL-33; FIGS. 12D and 12E). Similar to CD4⁺ T cells, the expression ratio of ST2 on splenic Tregs in the WT-PBS group was significantly higher than that in the ST2^{-/-}-PBS (P<0.05) and ST2^{-/-}-mIL-33 (P<0.05) groups, and tended to be lower than that in WT-mIL-33 group (FIGS. 12D and 12E). These results suggested that IL-33 protein might directly affect the immune-regulatory function of Tregs via ST2.

FIGS. 12A to 12E show that IL-33 protein exerts antitumor activity via ST2 and stimulates CD4⁺ T cells to express ST2. FIGS. 12A-12E show subcutaneous CT26 tumor-bearing mouse model. Mice were sacrificed on day 13 post PBS or mIL-33 treatment. FIG. 12A shows tumor volumes (left panel) and tumor weights (right panel). FIGS. 12B-12E show the flow-cytometric analysis of INF-γ and ST2 expression on splenic CD4⁺ T, CD8⁺ T, and NK cells, and TGF-β and ST2 expression on splenic Tregs. FIGS. 12B and 12D show representative dot plots. FIGS. 12C and 12E show quantitative data. Data are means±SD (n=5-6 mice per group). ns: no significant difference. *P<0.05, **P<0.01, ***P<0.001.

### 13. Endogenous IL-33 has No Effect on Tumor Growth and Immune Response

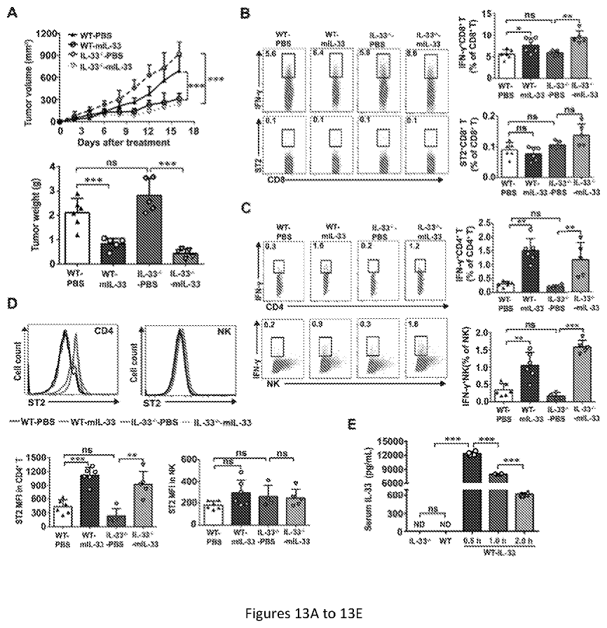

The above experiments showed that exogenous IL-33 protein had an antitumor immune effect; however, whether endogenous IL-33 protein has a similar effect remained unclear. In the MC38 cell subcutaneous tumor-bearing model, tumor mass did not significantly differ between the WT-PBS group and the IL-33^{-/-}-PBS (FIG. 13A, lower panel). However, tumor volume and mass were markedly reduced upon injection of exogenous mIL-33 protein (WT-PBS vs. WT-mIL-33, P<0.001; IL-33^{-/-}-PBS vs. IL-33^{-/-}-mIL-33, P<0.001; FIG. 13A). These results suggested that endogenous IL-33 protein might not have an effect on tumor growth.

In the spleen, the fraction of IFN-γ⁺CD8⁻T cells was not significantly different between the WT-PBS and IL-33^{-/-}-PBS groups, but was significantly lower than that in the corresponding mIL-33 group (WT-PBS vs. WT-mlL-33, P<0.05; IL-33^{-/-}-PBS vs. IL-33^{-/-}-mIL-33, P<0.01; FIG. 7B, upper panel). Additionally, neither endogenous nor exogenous IL-33 protein had a significant difference on ST2 expression by splenic CD8⁺ T cells (FIG. 13B, lower panel). In tumor tissues, the fraction of IFN-γ⁺CD4⁺ T cells was similar between the WT-PBS and IL-33^{-/-}-PBS groups, but was significantly increased upon injection of mIL-33 (WT-PBS vs. WT-mIL-33, P<0.01; IL-33^{-/-}-PBS vs. IL-33^{-/-}-mIL-33, P<0.01; FIG. 13C, upper panel). Similarly, the fraction of IFN-γ⁺NK cells was similar between the WT-PBS and IL-33^{-/-}-PBS groups, but was markedly elevated upon mIL-33 treatment (WT-PBS vs. WT-mIL-33, P<0.01; IL-33^{-/-}-PBS vs. IL-33^{-/-}-mIL-33, P<0.001; FIG. 13C, lower panel). These data suggested that endogenous IL-33 protein might not have a significant effect on the activation of CD4⁺ T, CD8⁺ T, and NK cells, which is very different from exogenous IL-33 protein.

Consistent with the results in ST2^{-/-} mouse experiments, exogenous mIL-33 protein significantly promoted ST2 expression on tumor-infiltrating CD4⁺ T cells (WT-PBS vs. WT-mIL-33, P<0.001; IL-33^{-/-}-PBS vs. IL-33^{-/-}-mIL-33, P<0.01; FIG. 13D), but did not affect ST2 expression on tumor-infiltrating NK cells (FIG. 13D). ST2 expression on tumor-infiltrating CD4⁺ T and NK cells was not significantly affected by depletion of endogenous IL-33 protein using IL-33^{-/-} mice (FIG. 13D). Serum levels of IL-33 were very low in IL-33^{-/-} and WT mice, but were greatly increased at 0.5 h, 1 h, and 2 h after mIL-33 injection (FIG. 13E). Endogenous IL-33 protein levels were very low and difficult to detect, and thus might not produce an immune response nor affect tumor growth and the expression of IFN-γ and ST2 on CD4⁺ T, CD8⁺ T, and NK cells.

FIGS. 13A to 13E show endogenous IL-33 protein cannot boost antitumor immunity. FIGS. 13A-13D show subcutaneous MC38 tumor-bearing mouse model. Mice were sacrificed on day 17 post PBS or mIL-33 treatment. FIG. 13A shows tumor volume (upper panel) and tumor weights (lower panel). FIG. 13B shows the flow-cytometric analysis of INF-γ and ST2 expression on splenic CD8⁺ T cells. Representative dot plots (left panel) and quantitative data (right panel). FIG. 13C shows the flow-cytometric analysis of INF-γ expression on CD4⁺ T and NK cells from tumors. Representative dot plots (left panel) and quantitative data (right panel). FIG. 13D shows the flow-cytometric analysis of ST2 expression on CD4⁺ T and NK cells from tumors. FIG. 13E shows serum levels of IL-33 in IL-33^{-/-}, WT, and WT-IL33 mice. Data are means±SDs (n=4-6 mice per group). ns: no significant difference. *P<0.05, **P<0.01, ***P<0.001.

### I. General Methods

Standard methods in Molecular Biology are known and used in the present disclosure. (See, e.g., Maniatis, et al., Molecular Cloning, A Laboratory Manual, Cold Spring Harbor Laboratory Press, Cold Spring Harbor, N.Y. (1982); Sambrook and Russell, Molecular Cloning, 3rd ed., Cold Spring Harbor Laboratory Press, Cold Spring Harbor, N.Y. (2001); Wu, Recombinant DNA, Vol. 217, Academic Press, San Diego, Calif. (1993)). Standard methods are also disclosed in Ausubel, et al., Current Protocols in Molecular Biology, Vols. 1-4, John Wiley and Sons, Inc. New York, N.Y. (2001), which describes cloning in bacterial cells and DNA mutagenesis (Vol. 1), cloning in mammalian cells and yeast (Vol. 2), protein expression (Vol. 3), and bioinformatics (Vol. 4).

Methods for protein purification including immunoprecipitation, chromatography, electrophoresis, centrifugation, and crystallization are known in the art. (See, e.g., Coligan, et al., Current Protocols in Protein Science, Vol. I, John Wiley and Sons, Inc., N.Y. (2000)). Chemical analysis, chemical modification, post-translational modification, and production of fusion proteins are known in the art too. (see, e.g., Coligan, et al., Current Protocols in Protein Science, Vol. 2, John Wiley and Sons, Inc., NY (2000); Ausubel, et al., Current Protocols in Molecular Biology, Vol. 3, John Wiley and Sons, Inc., NY, N.Y., pp. 16.0.5-16.22.17 (2001); Sigma-Aldrich, Co. Products for Life Science Research, St. Louis, Mo. (2001), pp. 45-89; Amersham Pharmacia Biotech, BioDirectory, Piscataway, N.J. (2001), pp. 384-391). Standard techniques for characterizing ligand/receptor interactions are known in the art. (See, e.g., Coligan, et al., Current Protocols in Immunology, Vol. 4, John Wiley, Inc., NY, (2001)).

Research models for the treatment and diagnosis of cancer are known in the art. (See, e.g., Alison (ed.), The Cancer Handbook, Grove's Dictionaries, Inc., St. Louis, Mo. (2001); Oldham (ed.), Principles of Cancer Biotherapy, 3rd ed., Kluwer Academic Publ., Hingham, Mass. (1998); Devita, et al. (eds.), Cancer: Principles and Practice of Oncology, 6th ed., Lippincott, Phila, P A (2001); Holland, et al. (eds.), Holland-Frei Cancer Medicine, BC Decker, Phila., Pa. (2000); Garrett and Sell (eds.), Cellular Cancer Markers, Humana Press, Totowa, N.J. (1995); MacKie, Skin Cancer, 2nd ed., Mosby, St. Louis (1996); Moertel, New Engl. J. Med. 330:1136-1142 (1994); Engleman, Semin. Oncol. 30 (3 Suppl. 8):23-29 (2003); Mohr, et al., Onkologie 26:227-233 (2003)).

### II. Materials and Methods

### Mice

BALB/c (wild-type, WT) and C57BL/6 (wild-type, WT) mice were obtained from SLAC Lab Animal (Shanghai, China). ST2^{-/-} mice (BALB/c background), originally obtained from Medical Research Council Laboratory of Molecular Biology (Cambridge, UK), were kindly provided by Dr. YanQing Wang, School of Basic Medical Sciences, Fudan University (Shanghai, China). IL-33^{-/-} mice (C57BL/6 background) were obtained from Shanghai Model Organisms Center (Shanghai, China). Six-to-eight-week-old male mice were used in all experiments. All animal experiments were authorized by the Animal Care and Use Committee of Shanghai Jiao Tong University (Shanghai, China).

### Tumor Cells

CT26 colon carcinoma cells were purchased from ATCC (Rockville, Md., USA) and were cultured in RPMI 1640 complete medium (containing 10% fetal bovine serum, FBS). MC38 colon adenocarcinoma cells were obtained from Biovector NTCC (Beijing, China) and were maintained in DMEM complete medium (containing 10% FBS). RPMI 1640, DMEM, and FBS were purchased from Gibco (Grand Island, USA).

Expression, Purification, Identification, and Bioactivity Assay of mIL-33 and hIL-33

The coding sequence of mature mIL-33 and mature hIL-33 was optimized and subcloned into the expression vector pET-43.1a (+), and then transformed into BL21 to express, respectively. In order to acquire high expression levels of soluble IL-33, the expression condition of induced-temperature and induced-time were optimized. Finally, expression was induced with 1 mM IPTG (Sigma-Aldrich, USA) at 25° C. for 6 h. The theoretical isoelectric point of mature mIL-33 (Ser 109-Ile 266) and mature hIL-33 (Ser 112-Thr270) was 4.52 and 4.80, respectively, which both belong to acid proteins. Therefore, we firstly utilized anion-exchange chromatography (Q Sepharose™ Fast Flow) to separate mIL-33 and hIL-33. In order to further purification, we used gel filtration (Superdex 26/60 75 μg) and acquired a large quantity of target proteins whose purity was more than 90%.

Subsequently, the specificity of target proteins was determined by western blot. Purified mIL-33 and hIL-33 can specifically bind mouse natural soluble receptor ST2 fusion protein (mST2-Fc, BioLegend, San Diego, Calif., USA). In addition, the affinity analysis of mIL-33 and hIL-33 binding to mST2-Fc using ELISA was performed. The detection values of wells coated with mIL-33 or hIL-33 were gradually increased with the increasing concentration of mST2-Fc, but detection values of wells coated with 1% BSA had no significant change. The above data suggested that target proteins without any purification tag were successfully obtained.

IL-33 can induce Raw264.7 mouse macrophage cells and P815 mouse mastocytoma cells to secrete mTNF-α and mIL-6, respectively. Based on this finding, bioactivity analysis of purified mIL-33 and hIL-33 was carried out. The EC50 value of mTNF-α and mIL-6 induced by mIL-33 was 10.0 ng/mL and 1.5 ng/mL, respectively. The EC50 value of mTNF-α and mIL-6 induced by hIL-33 was 801.0 ng/mL and 392.7 ng/mL, respectively. These data suggested that the pure mIL-33 and hIL-33 were both biologically active.

Recombinant Mouse IL-33 (mIL-33) Production and Bioactivity Analysis

Purified mIL-33 protein was identified by western blotting and enzyme-linked immunoassay (ELISA).

To detect the biological activity of mIL-33, 5×10⁴ Raw264.7 and 4×10³ P815 cells (both from Stem Cell Bank, Chinese Academy of Sciences, China) were seeded into each well of 96-well plates, respectively. The plates were left to stand for 1 hour. Then, the supernatant was discarded and 200 μL of RPMI 1640 (Raw264.7) or DMEM (P815) complete medium containing various concentrations of mIL-33 was added. After incubation at 37° C. in the presence of 5% CO₂ for 18 hours (Raw264.7) or 48 hours (P815), cell culture supernatants were collected and used to assess mouse TNF-α (Raw264.7) and IL-6 (P815) expression by ELISA (R&D Systems, Minneapolis, Minn., USA).

Mouse Tumor Models and mIL-33 Treatments

To establish subcutaneous tumor-bearing mouse models, BALB/c or ST2^{-/-} mice were inoculated subcutaneously with 1×10⁶ CT26 cells, and C57BL/6 or IL-33^{-/-} mice were injected subcutaneously with 2×10⁶ MC38 cells. Tumor volume (mm³) was monitored every two days from the day they were visible. Mice were sacrificed at 2 to 4 weeks after the tumor inoculation. Tumors were collected and weighed. For the induction of pulmonary metastasis, 3×10⁵ CT26 cells in 100 μL PBS were intravenously (i.v.) injected into the tail vein of BALB/c mice. In the liver metastasis model, 5×10⁴ CT26 cells in 50 μL PBS were injected into the splenic capsule of BALB/c mice. The extent of metastasis was assessed by comparing the numbers of visible tumor nodules or hematoxylin & eosin (H&E) staining on day 14 (lung) or 17 (liver) after CT26 cell inoculation.

In the subcutaneous tumor-bearing mouse model, two administration methods for mIL-33 treatments were tested. Firstly, 100 μg/kg mIL-33 was injected subcutaneously into mice, twice daily, starting on day 0 up to day 14 after tumor-cell inoculation (FIG. 1A). Secondly, 90 μg/kg mIL-33 was injected subcutaneously into mice, once daily, starting on day 5 (visible tumor) after tumor-cell inoculation up to the end of the test. In subsequent experiments (FIGS. 2-7), the second method was administered.

For the pulmonary metastasis model, mIL-33 (100 μg/kg) was injected subcutaneously into mice, twice daily, starting on the inoculation day. For the liver metastasis model, mice were injected subcutaneously with 100 μg/kg mIL-33 (twice daily) on day 8 post inoculation. Delaying the initial administration time mainly prevented the mice from scratching the wound (spleen inoculation of tumor cells requires cutting the skin and stitching), which can cause infection.

### Antibodies and Flow-Cytometric Analysis

The following fluorochrome-conjugated anti-mouse antibodies were used for flow cytometry: anti-CD16/32 (2.4G2), anti-CD3 (145-2C11), anit-CD49b (DX5), anti-CD8 (53-6.7), anti-CD4 (GK1.5), anti-CD25 (PC61.5), anti-CD45 (30-F11), anti-CD69 (H1.2F3), anti-Foxp3 (FJK-16s), anti-T-bet (4610), anti-Fomes (Dan11mag), anti-PD-1 (RMP1-30), anti-Grl (RB6-8C5), anti-Siglec-F (E50-2440), anti-CD11b (M1/70), anti-CD11c (N418), anti-F4/80 (BM8), anti-CD40 (3/23), anti-CD40L (MR1), anti-MHCII (M5/114.15.2), anti-IFN-γ (XMG1.2), anti-TGF-β (TW7-16134), and anti-ST2 (DIH9). These antibodies and their matched isotype controls were purchased from BD Biosciences (Franklin Lakes, N.J., USA), eBioscience (San Diego, Calif., USA), or BioLegend (San Diego, Calif., USA).

Single-cell suspensions from spleen and tumor tissues was prepared. For intracellular staining, a transcription factor buffer set (BD Biosciences) was used according to the manufacturer's instructions. For IFN-γ, TGF-β, and CD40L detection, the Cell Stimulation Cocktail (plus protein transport inhibitors) (Invitrogen, Carlsbad, Calif., USA) was used. Flow cytometry and data analysis were conducted using an LSRFortessa™ instrument (BD Biosciences) and FlowJo (Tree Star Inc., Ashland, Oreg., USA), respectively.

### Depletion of Cells In Vivo and Quantitative Reverse Transcription (RT-q) PCR

For depletion of CD4⁺ T cells or Tregs (CD4⁺CD25⁺Foxp3⁺), mice were given intraperitoneal injections of 200 μg anti-CD4 (GK1.5, BioXcell, West Lebanon, N.H., USA) or anti-CD25 (PC-61.5.3, BioXcell) every 3 days. Depletion of eosinophils was achieved by intraperitoneal injections of 15 μg anti-Siglec-F (MA617061, R&D Systems) every other day. IgG2b (LTF-2, BioXcell) was used as the isotype control and all antibodies, dissolved in PBS, were injected on the day before mIL-33 treatment.

Total RNA was extracted from CT26 tumor tissues in isotype (IgG2b)+PBS group and isotype+mIL-33 group with TRIzol reagent (Invitrogen) and was reverse transcribed using PrimeScript™ RT Master Mix (Takara, Dalian, China) (n=4 mice per group). Primers for RT-qPCR (see supplementary Table 1) were synthesized by Invitrogen (Shanghai, China). Relative mRNA levels were conducted three times independently on an Applied Biosystems StepOnePlus instrument using TB Green Premix Ex TagII (Takara, Dalian, China). GAPDH was used as a reference gene. Relative mRNA levels were determined using the 2^{-ΔΔct} method.

### Monoclonal Antibody Blocking Experiments

For blocking of CD40/CD40L signaling pathway, mice were given intraperitoneal injections of 200 μg anti-CD40 (MR-1, BioXcell, West Lebanon, N.H., USA) every 3 days. Hamster IgG (hamster IgG f(ab′)2 fragment, BioXcell) was used as the isotype control, and all antibodies, dissolved in PBS, were injected on the day before mIL-33 treatment.

### ELISA of Serum Levels of IL-33

Serum levels of IL-33 in IL-33^{-/-} mice, wild-type (WT, C57BL/6) mice, and IL-33-administrated (WT-IL-33) mice were measured using a mouse IL-33 ELISA kit (R&D Systems) according to the manufacturer's instructions. WT-IL-33 mice were treated subcutaneously with 90 μg/kg mIL-33, sacrificed after 0.5, 1, and 2 h, and serum was collected.

### Statistical Analysis

Data are presented as the mean±standard deviation (SD). Two-tailed Student's unpaired t-test was used to compare means between two groups (numbers of metastasis foci or cells, tumor weight, serum levels of IL-33, and the cell ratio or mean fluorescence intensity (MFI) in flow-cytometric analysis). Repeated-measures ANOVA was used to compare tumor volumes among groups. P<0.05 was considered statistically significant. All data were processed in SPSS v.18.0 (IBM, Armonk, N.Y., USA) or GraphPad Prism 5 Software (San Diego, Calif., USA).

What is claimed:

1. A method of treating, preventing, or reducing onset or metastasis of a cancer, comprising administering to a subject in need a therapeutically effective amount of human IL-33 protein or a polypeptide having a corresponding sequence substantially identical thereto.

2. The method of claim 1, wherein the IL-33 protein is human IL-33.

3. The method of claim 2, wherein the human IL-33 is recombinant human IL-33.

4. The method of claim 2, wherein the human IL-33 has a sequence of SEQ ID NO:1.

5. The method of claim 1, wherein the subject is human.

6. The method of claim 1, wherein the cancer is selected from the group consisting of a solid tumor selected from pancreatic cancer, small cell lung cancer (SCLC), hepatocellular carcinoma (HCC), squamous cell carcinoma, non-small cell lung cancer, squamous non-small cell lung cancer (NSCLC), non-squamous NSCLC, glioma, gastrointestinal cancer, renal cancer, ovarian cancer, liver cancer, colorectal cancer, endometrial cancer, kidney cancer, prostate cancer, thyroid cancer, neuroblastoma, glioblastoma, stomach cancer, bladder cancer, hepatoma, breast cancer, colon carcinoma, head and neck cancer, gastric cancer, germ cell tumor, pediatric sarcoma, sinonasal natural killer, melanoma, skin cancer, bone cancer, cervical cancer, uterine cancer, carcinoma of the fallopian tubes, carcinoma of the endometrium, carcinoma of the cervix, carcinoma of the vagina, carcinoma of the vulva, cancer of the anal region, testicular cancer, cancer of the esophagus, cancer of the small intestine, cancer of the endocrine system, cancer of the parathyroid gland, cancer of the adrenal gland, sarcoma of soft tissue, cancer of the urethra, cancer of the ureter, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus-related cancers, and cancers of viral origin; or a hematological cancer selected from acute lymphoblastic leukemia (ALL), acute myelogenous leukemia (AML), chronic lymphocytic leukemia (CLL), chronic myelogenous leukemia (CML), Hodgkin's lymphoma (HL), non-Hodgkin's lymphomas (NHLs), multiple myeloma, smoldering myeloma, monoclonal gammopathy of undetermined significance (MGUS), advanced, metastatic, refractory and/or recurrent hematological malignancies, and any combinations of said hematological malignancies.

7. The method of claim 6, wherein the cancer is selected from the group consisting of hepatocellular carcinoma (HCC), lung cancer preferably LLC (Lewis lung carcinoma), gastric cancer, colon cancer, and prostate cancer.

8. The method of claim 7, wherein the cancer is hepatocellular carcinoma (HCC).

9. The method of claim 7, wherein the cancer is lung cancer.

10. The method of claim 9, wherein the lung cancer is Lewis lung carcinoma.

11. The method of claim 7, wherein the cancer is gastric cancer.

12. The method of claim 1, further comprising administering with at least one anticancer entity.

13. The method of claim 12, wherein the anticancer entity is selected from the group consisting of a cytokine, an immunocytokine, TNFα, a PAP inhibitor, an oncolytic virus, a kinase inhibitor, an ALK inhibitor, a MEK inhibitor, an IDO inhibitor, a GLS1 inhibitor, a tyrosine kinase inhibitor, a CART cell or T cell therapy, a TLR agonist, or a tumor vaccine, or an antibody selected from the group consisting of an anti-CTLA-4 antibody, an anti-CD3 antibody, an anti-CD4 antibody, an anti-CD8 antibody, an anti-4-1 BB antibody, an anti-PD-1 antibody, an anti-PD-L1 antibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSF1 R antibody, an anti-CSF1 antibody, an anti-IL-7R antibody, an anti-MARCO antibody, an anti-CXCR4 antibodies, an anti-VEGF antibody, an anti-VEGFR1 antibody, an anti-VEGFR2 antibody, an anti-TNFR1 antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD 52 antibody, an anti-CCR4 antibody, an anti-CCR8 antibody, an anti-CD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRG1 antibody, an anti-FLT3 antibody, an anti-B7-H4 antibody, an anti-B7-H3 antibody, an KLRG1 antibody, a BTN1A1 antibody, and an anti-GITR antibody.

14. A composition comprising human IL-33 protein or a polypeptide having a corresponding sequence substantially identical thereto as active ingredient and at least one pharmaceutically acceptable carrier for use in treatment, prevention or reduction of onset or metastasis of a cancer.

15. The method of claim 14, wherein the IL-33 protein is human IL-33 protein.

16. A method of treating, preventing, or reducing onset or metastasis of a cancer, comprising administering to a subject in need a therapeutically effective amount of an agent capable of upregulating CD40/CD40L signaling pathway, or a polypeptide having a corresponding sequence substantially identical thereto.

17. The method of claim 16, wherein the agent capable of upregulating CD40/CD40L signaling pathway is IL-33 protein.

18. The method of claim 17, wherein the IL-33 protein is human IL-33 protein.

19. The method of claim 18, wherein the human IL-33 is recombinant human IL-33.

20. The method of claim 16, wherein the subject is human.

21. The method of claim 16, wherein the cancer is selected from the group consisting of the group consisting of a solid tumor selected from pancreatic cancer, small cell lung cancer (SCLC), hepatocellular carcinoma (HCC), squamous cell carcinoma, non-small cell lung cancer, squamous non-small cell lung cancer (NSCLC), non-squamous NSCLC, glioma, gastrointestinal cancer, renal cancer, ovarian cancer, liver cancer, colorectal cancer, endometrial cancer, kidney cancer, prostate cancer, thyroid cancer, neuroblastoma, glioblastoma, stomach cancer, bladder cancer, hepatoma, breast cancer, colon carcinoma, head and neck cancer, gastric cancer, germ cell tumor, pediatric sarcoma, sinonasal natural killer, melanoma, skin cancer, bone cancer, cervical cancer, uterine cancer, carcinoma of the fallopian tubes, carcinoma of the endometrium, carcinoma of the cervix, carcinoma of the vagina, carcinoma of the vulva, cancer of the anal region, testicular cancer, cancer of the esophagus, cancer of the small intestine, cancer of the endocrine system, cancer of the parathyroid gland, cancer of the adrenal gland, sarcoma of soft tissue, cancer of the urethra, cancer of the ureter, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus-related cancers, and cancers of viral origin; or a hematological cancer selected from acute lymphoblastic leukemia (ALL), acute myelogenous leukemia (AML), chronic lymphocytic leukemia (CLL), chronic myelogenous leukemia (CML), Hodgkin's lymphoma (HL), non-Hodgkin's lymphomas (NHLs), multiple myeloma, smoldering myeloma, monoclonal gammopathy of undetermined significance (MGUS), advanced, metastatic, refractory and/or recurrent hematological malignancies, and any combinations of said hematological malignancies.

22. The method of claim 21, wherein the cancer is selected from the group consisting of hepatocellular carcinoma (HCC), lung cancer, gastric cancer, colon cancer, and prostate cancer.

23. The method of claim 22, wherein the cancer is hepatocellular carcinoma (HCC).

24. The method of claim 22, wherein the cancer is lung cancer.

25. The method of claim 24, wherein the lung cancer is Lewis lung carcinoma.

26. The method of claim 22, wherein the cancer is gastric cancer.
